(19) United States
(12) Patent Application Publication
(10) Pub. No.: US 2024/0294862 A1
(43) Pub. Date: Sep. 5, 2024

(54) CONTROL OF pH IN PARALLEL CULTURE WELLS

(71) Applicant: Beckman Coulter, Inc., Brea, CA (US)

(72) Inventor: Kai Uwe Neumann, Aachen (DE)

(73) Assignee: Beckman Coulter, Inc., Brea, CA (US)

(21) Appl. No.: 18/559,296

(22) PCT Filed: May 2, 2022
(86) PCT No.: PCT/US2022/027348

§ 371 Date: Nov. 6, 2023

Related U.S. Application Data
(60) Provisional application No. 63/185,779, filed on May 7, 2021.

(51) Int. Cl. C12M 1/36; C12M 1/00; C12M 1/32; C12M 1/34; C12M 3/06

(52) U.S. Cl. CPC C12M 41/48; C12M 23/12; C12M 23/16; C12M 23/38; C12M 41/26

(57) ABSTRACT

A workflow and associated system for controlling pH is described that allows for real-time pH adjustment in parallel culture wells of a microplate. For example, a biological process control system comprised of a pH measurement system, a controller, and a pH adjustment system implements a closed control loop, where a pH of a culture well measured by the pH measurement system is compared to a predetermined pH for the culture well, and, if there is a deviation, a pH adjustment system adjusts the pH in the culture well to correct the deviation as the system continues on to measure, evaluate, and adjust, if needed, remaining microplate culture wells. Therefore, by performing per-well pH evaluation and adjustment in real-time upon receiving the measured pH rather than waiting until pH of all culture wells are measured, the measured pH used as a basis for pH adjustment is a current, accurate measurement.

### CROSS-REFERENCE TO RELATED APPLICATIONS

This application claims the benefit of and priority to U.S. Provisional Patent Application No. 63/185,779, filed May 7, 2021, the entire contents of which are incorporated herein by reference.

### BACKGROUND

In many areas of biology, process technology, pharmacy and medicine, there is a need for high throughput screening of biological systems for process development, fed-batch optimization, and media screening, among other examples, which may be achieved via parallelization of experiments. A microplate including a plurality of individual wells is one example device utilized to achieve a high number of parallel operations. As one example, for cell culture, each of the individual culture wells can be filled with medium, inoculated to introduce cells into the medium, and incubated at a particular temperature using a shaking incubator. Process parameters, including a pH value, can be continuously measured for each individual culture well during the growth process.

The pH in the culture well is an important environmental influence for cell growth. The activities of the enzymes that catalyze reactions due to the metabolic process of the cells are decisively influenced by the pH value. However, the pH value of the medium continuously changes as a result of the metabolism of the cells and as a result of the consumption of the components of the culture medium. If pH control is lacking, it can be difficult to achieve high cell densities. Therefore, accurate control of pH value is desirable.

### SUMMARY

In general terms, this disclosure is directed to the control of pH in parallel culture wells. In one possible configuration and by non-limiting example, once pH in a culture well is measured, pH control for that culture well can be initiated before pH in a next culture well is measured (e.g., rather than waiting for pH in each of the culture wells to be measured before initiating the pH control). The pH control can include a comparison of a measured pH value to a predetermined pH value for the culture well to determine whether there is a deviation, and if there is a deviation, the pH control can further include an adjustment to the pH in the culture well.

In one aspect, an example system for controlling pH in parallel culture wells is described. The example system includes a pH measurement system that, during a cycle, successively measures pH for each of a plurality of culture wells contained in a microplate, a pH adjustment system that, during the cycle, adjusts the pH in one or more of the plurality of culture wells, and a controller communicatively coupled to the pH measurement system and the pH adjustment system. The controller includes a processing device and a memory coupled to the processing device. The memory stores instructions that, when executed by the processing device, causes the controller to receive, from the pH measurement system, a measured pH value for a first culture well of the plurality of culture wells, and initiate pH control for the first culture well before receiving, from the pH measurement system, one or more additional measured pH values for one or more other culture wells of the plurality of culture wells. The pH control includes comparing the measured pH value in the first culture well to a predetermined pH value for the first culture well, based on the comparison, determining whether the measured pH value deviates from the predetermined pH value, and in response to a determination that the measured pH value deviates from the predetermined pH value, generating and providing a signal to the pH adjustment system to cause the pH adjustment system to adjust the pH in the first culture well to correct the deviation

In another aspect, an example method for controlling pH in parallel culture wells is described. The example method includes measuring pH in a culture well of a plurality of culture wells contained in a microplate, and initiating pH control for the culture well before measuring pH in at least one or more other culture wells of the plurality of culture wells. The pH control includes comparing the measured pH in the culture well to a predetermined pH for the culture well, based on the comparison, determining whether the measured pH deviates from the predetermined pH, and in response to a determination that the measured pH deviates from the predetermined pH, adjusting the pH in the culture well to correct the deviation.

In a further aspect, example computer readable non-transitory storage media are described. The computer readable non-transitory storage media store instructions that, when executed by at least one processing device, cause the at least one processing device to receive, from a pH measurement system, a measured pH value for a culture well of a plurality of culture wells contained in a microplate, and initiate pH control for the culture well before receiving, from the pH measurement system, one or more additional measured pH values for one or more other culture wells of the plurality of culture wells. The pH control further causes the at least one processing device to compare the measured pH value in the culture well to a predetermined pH value for the culture well, based on the comparison, determine whether the measured pH value deviates from the predetermined pH value, and in response to a determination that the measured pH value deviates from the predetermined pH value, generate and provide a signal to a pH adjustment system to cause the pH adjustment system to adjust the pH in the culture well to correct the deviation.

### BRIEF DESCRIPTION OF THE DRAWINGS

FIG. 1 depicts an example bioprocess control system capable of real-time pH adjustment.

FIG. 2 is a block diagram of example components of the bioprocess control system described in FIG. 1.

FIG. 3 is an example computing device of the bioprocess control system described in FIG. 1 and FIG. 2.

FIG. 4 is a flowchart illustrating a method for controlling pH in parallel culture wells.

FIG. 5A is an example microplate configuration and associated pH measurement pattern.

FIG. 5B is another exemplary pH measurement pattern for the microplate configuration shown in FIG. 5A.

FIG. 6 is another example microplate configuration and associated pH measurement pattern.

FIG. 7 is a conceptual diagram illustrating a pH measurement of an individual culture well of a microplate.

FIG. 8 is a conceptual diagram illustrating a closed control loop implemented by the bioprocess control system for pH control.

FIG. 9 illustrates an example configuration for components of the pH adjustment system to enable pH adjustment of the culture wells.

FIG. 10 illustrates another example configuration for components of the pH adjustment system to enable pH adjustment of the culture wells.

FIG. 11 illustrates an example pH adjustment system integrated with a microplate.

FIG. 12 is a conceptual diagram illustrating pneumatically controlled valves of an example pH adjustment system.

FIG. 13 is a conceptual diagram illustrating an example sequence for controlling pH in real time for each of a plurality of microplate wells over a cycle.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

When using an instrument, such as a bioreactor or microbioreactor, to control bioprocesses in culture wells of a microplate placed therein, process parameters, including pH, can be measured for each culture well in the microplate. As discussed briefly above, pH of a culture well is an important environmental influence for cell growth that continuously changes, and therefore accurate pH control of the culture well is desirable. In many conventional workflows, the pH along with one or more other process parameters is measured from each of the individual culture wells (e.g., sequentially in a measurement pattern from one well to a next well) in a cycle, and any necessary pH adjustments to the culture wells based on the pH measurements can be made after pH has been measured for all of the culture wells in the cycle. As one illustrative example, one cycle may take about 3 minutes to run when three process parameters are being read for each of 32 wells in a microplate. However, because pH in the well can fluctuate within a short period of time, when a workflow of this type that waits for an entire cycle to be completed prior to adjusting the pH, deficiencies in the pH control can result. For example, a pH value of a culture well measured at a beginning of a cycle (e.g., within the first few seconds of the 3-minute cycle) is likely no longer representative of the pH of that culture well by the end of the cycle due to fluctuations caused by the metabolism of the cells and as a result of the consumption of the components of the medium, for example. Therefore, using the measured pH value, which is no longer representative of the pH in the culture well, as a basis for the pH adjustment performed after the cycle has completed can lead to inaccuracies. These inaccuracies are particularly impactful for certain type of cells that require a very narrow pH range for optimal growth.

To prevent such inaccuracies, an improved workflow and associated system for controlling pH is described herein to allow for real-time pH adjustment in parallel culture wells (e.g., two or more culture wells in a microplate that are being used concurrently for cell culture). For example, as part of a closed control loop, immediately after a pH measurement system measures pH of a culture well, pH control can be initiated by a controller to compare the measured pH to a predetermined pH for the culture well and cause a pH adjustment system to adjust the pH in the culture well to correct a deviation determined based on the comparison. For example, the culture well can be dosed with pH adjusting fluid to correct the deviation. In some examples, the dosing of the culture well with the pH adjusting fluid can occur within about 10 seconds of the pH being measured. Therefore, the measured pH used as a basis for the pH adjustment is a current, accurate measurement of pH value in the culture well such that the dosing of the culture well with the pH adjusting fluid will conform to the predetermined pH. Additionally, as one or more aspects of the pH control are being performed with respect to the culture well, pH measurement can be concurrently performed for a next culture well in the measurement pattern sequence (e.g., pH measurement and pH control are run in parallel).

To enable the pH measurement and pH control to run in parallel, the controller may be a controller with higher computing power capable of running an operating system that directly manages hardware and resources, like a processing device, memory, and storage. This is in contrast to the low power microcontrollers often used to implement the conventional workflows that do not allow for parallel computing (e.g., are not capable of controlling the pH of each well after a measurement while continuing to measure remaining wells). For example, using the low power microcontroller to perform pH control after each pH measurement, a workflow would be limited to having to measure pH of a first well and control the pH of the first well in its entirety before moving to a next well to measure and control the pH of the remaining wells in a sequential manner, which would greatly delay the cycle (e.g., increase an amount of time it takes to measure and control pH in each of the wells).

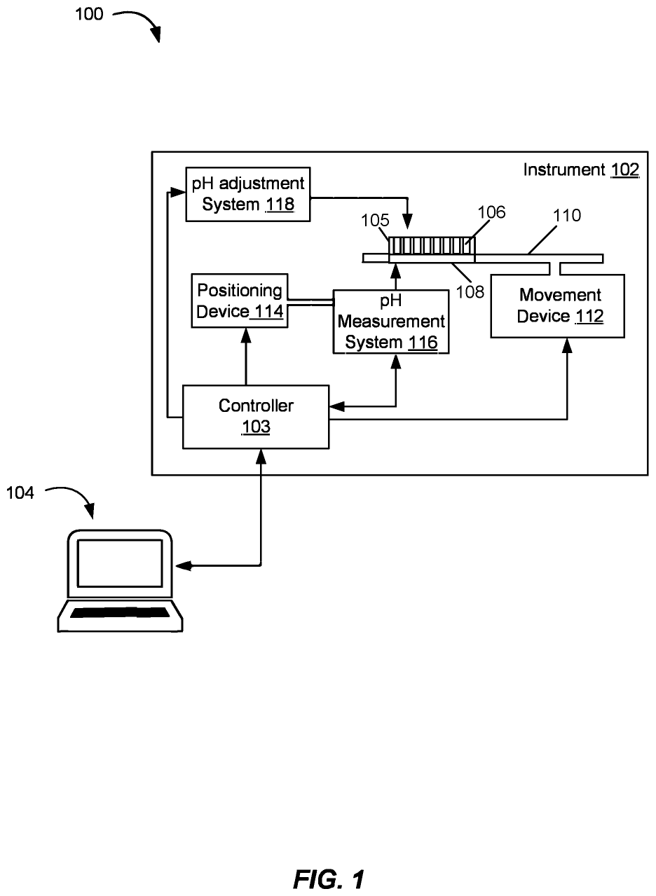

FIG. 1 depicts an example bioprocess control system 100 capable of real-time pH adjustment, hereinafter referred to as system 100. The system 100 includes an instrument 102 and a computing device 104 communicatively coupled to at least one or more components of the instrument 102, including at least a controller 103 of the instrument 102. In some examples, the instrument 102 is a bioreactor or a microbioreactor used to facilitate the control of bioprocesses for a variety of purposes, including for process development, fed-batch optimization, media screening, pH profiling, or induction profiling, among other examples. A microplate 105 can be inserted into the instrument and placed onto a support coupled to a movement device 112. The movement device 112 can include a mechanical shaker (e.g., an orbital shaker), a stirrer, or an ultrasound device, among other examples, that is communicatively coupled to and controlled by the controller 103.

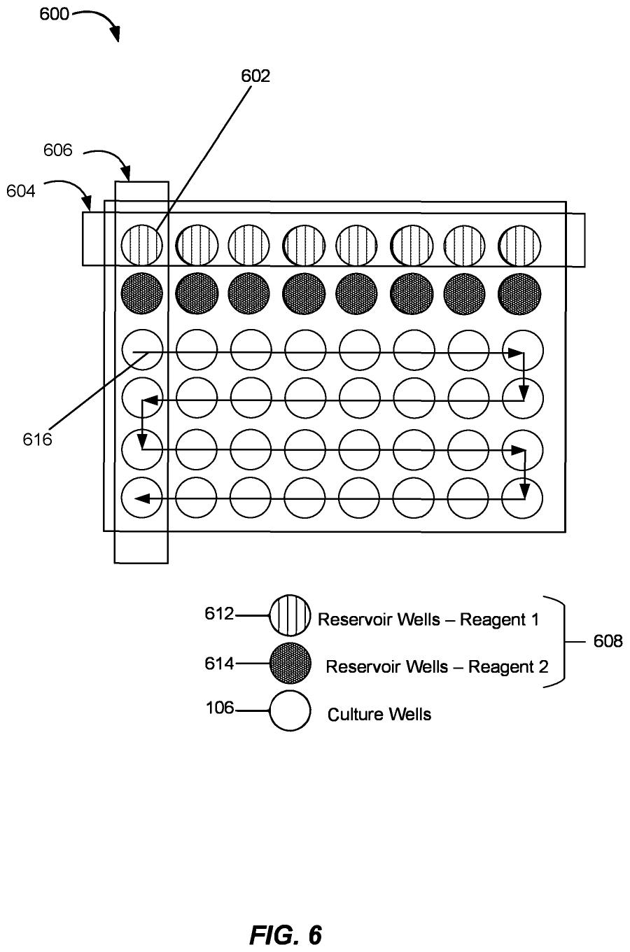

The microplate 105 can include a plurality of culture wells 106. In some examples, the culture wells 106 are filled with medium that includes various nutrients to promote microorganism development, inoculated to introduce microorganisms (e.g., a single cell or colony of cells) into the medium, and incubated at particular temperatures while being shaken by the movement device 112. Example microorganisms introduced can include bacteria, yeast, fungi, plant cells, insect cells, or mammalian cells. Additionally, aerobic, anaerobic, or microaerophilic cultures can be supported. In some examples, and as described in more detail with respect to FIG. 6, the microplate 105 can also include reservoir wells that contain various solutions that may be fed or dosed into the culture wells 106, including nutrient solutions to promote microorganism development and pH adjusting solutions to enable pH control. In other examples, these types of solutions may be contained in reservoirs separate from the microplate 105.

A bottom surface 108 of the microplate 105 and at least the culture wells 106 contained therein can be comprised of a transparent membrane that is permeable to electromagnetic radiation (e.g., light) to enable optical measurement of various process parameters, including pH, dissolved oxygen (DO), biomass, and fluorescence intensity of fluorescing molecules or proteins. Additionally, in some examples, a membrane or a lid, may seal or cover a top surface of the microplate 105 and the culture wells 106 contained therein to enable a sterile or monoseptic environment. When a seal or lid is used, additional structures such as apertures can be included to allow samples to be automatically extracted via the top surface of the wells, as well as to allow solutions (e.g., nutrient solutions or pH adjusting solutions) to be introduced over the top surface of the wells.

The instrument 102 also includes a pH measurement system 116 and a pH adjustment system 118 that form a closed control loop with the controller 103 to control the pH while an experiment is run by the instrument 102 in accordance with a protocol. For example, the controller 103 successively receives measured pH values for each culture well 106 from the pH measurement system 116 as it is being moved well-to-well via a positioning device 114, determines whether pH adjustment is needed based on those received pH measurements, and if pH adjustment is needed, causes the pH adjustment system 118 to adjust the pH in the respective well in real-time.

In some examples, the positioning device 114, the pH measurement system 116, and the pH adjustment system 118 are communicatively coupled to and controlled by the controller 103. Alternatively, in other examples, the positioning device 114, the pH measurement system 116, and the pH adjustment system 118 can be communicatively coupled to and controlled directly by the computing device 104 (e.g., based on signals received directly at the components from the computing device 104) and/or by the computing device 104 via the controller 103 (e.g., based on signals intercepted by the controller 103 from the computing device 104 and provided to the components). In some examples, these components are coupled to the computing device 104 via a wired connection (e.g., via Ethernet). In other examples, the connection can be wireless over a network, such as the Internet.

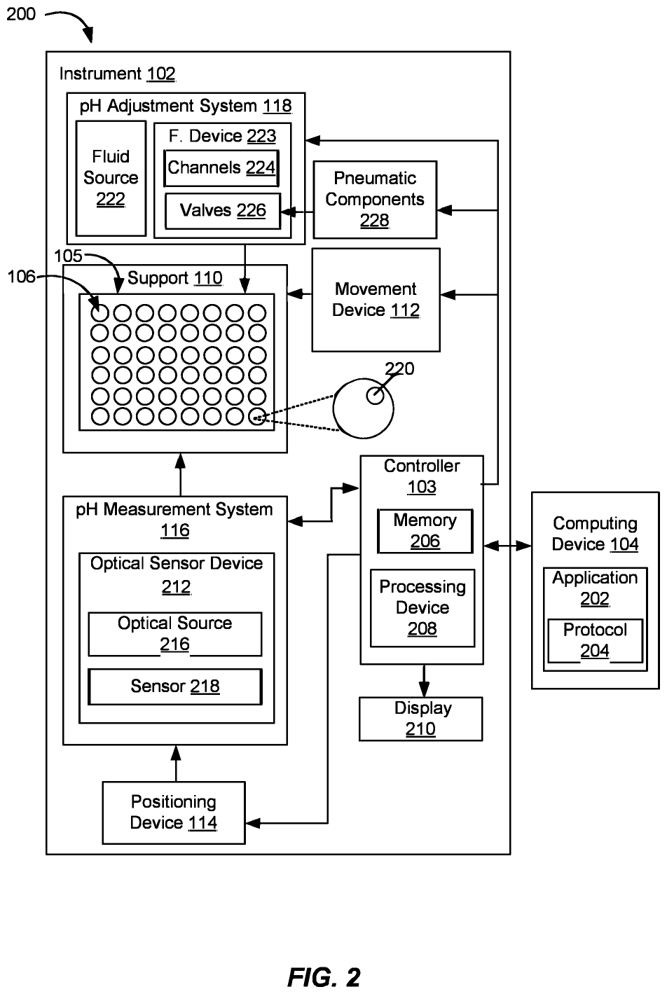

As described in more detail with reference to FIG. 2, the protocol can include requirements to measure pH of at least a subset of the culture wells 106 in the microplate 105. When the experiment is run by the instrument 102 in accordance with the protocol, the positioning device 114 can be controlled by the computing device 104 to successively position the pH measurement system 116 from well-to-well in a predefined pattern or sequence every set interval of time. The pH measurement system 116 can be positioned via an arm or support of the positioning device 114 to which the pH measurement system 116 is attached. In some examples, the positioning device 114 is an X-Y axes system.

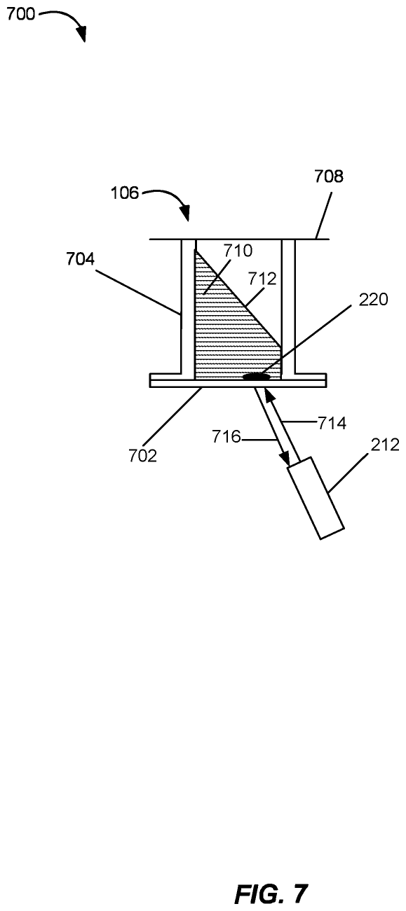

The pH measurement system 116 can be an optical measurement system that utilizes electromagnetic radiation (e.g., light) for measuring process parameters such as pH. In addition to pH, other process parameters including DO, biomass, and fluorescence intensity of fluorescing molecules or proteins can be measured by such an optical measurement system. Accordingly, the positioning device 114 positions the pH measurement system 116 in alignment with a culture well 106 underneath the microplate 105 given the light permeable membrane forming the bottom surface 108. The pH measurement system 116 can then be controlled by the computing device 104 to measure the pH in the culture well 106 as described in detail with reference to FIG. 7. In some examples, one or more of the other process parameters can also be measured in the culture well 106 before the pH measurement system 116 is positioned by the positioning device 114 to align with a next culture well 106 in the predefined measurement pattern or sequence.

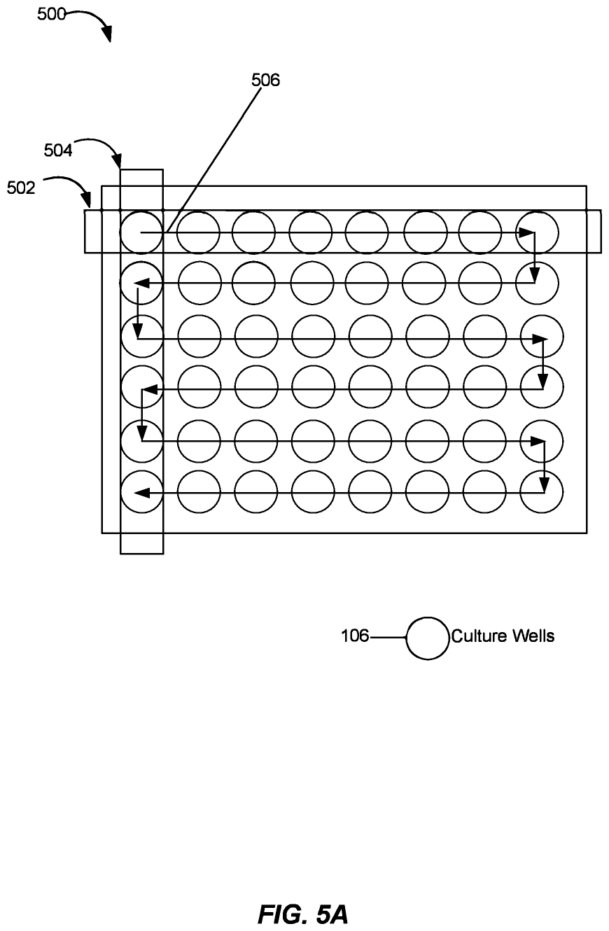

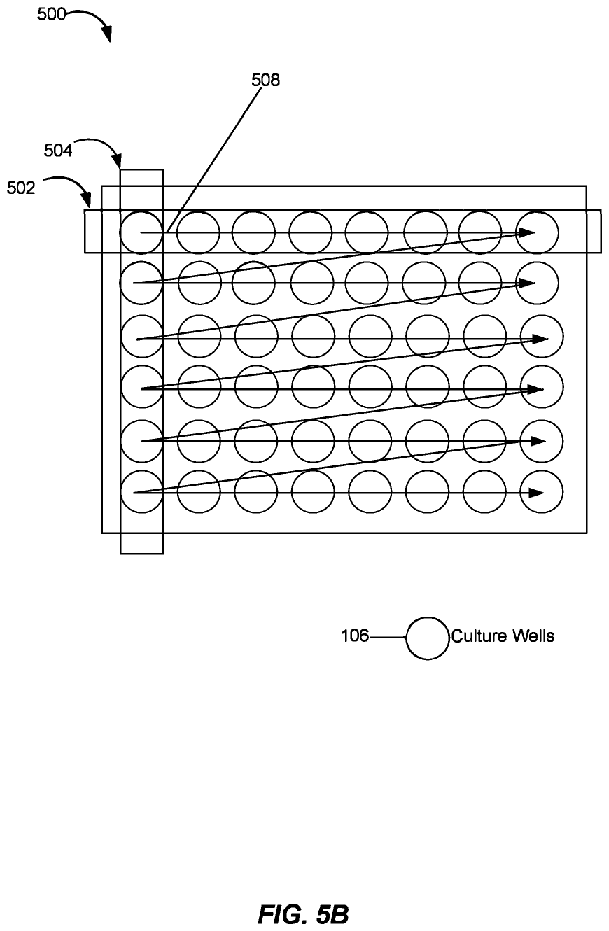

Example predefined measurement patterns or sequences by which the pH measurement system 116 is moved from well-to-well by the positioning device 114 is shown in FIGS. 5A, 5B, and 6 below. In one example, the interval of time between which the pH measurement system 116 is moved from well-to-well by the positioning device 114 is at least long enough to allow for the pH measurement system 116 to measure pH in the current culture well 106 with which the pH measurement system 116 is aligned prior to the positioning device 114 moving the pH measurement system 116 in alignment with a next culture well 106. In another example, the interval of time is further extended to allow for measurement of the one or more additional process parameters.

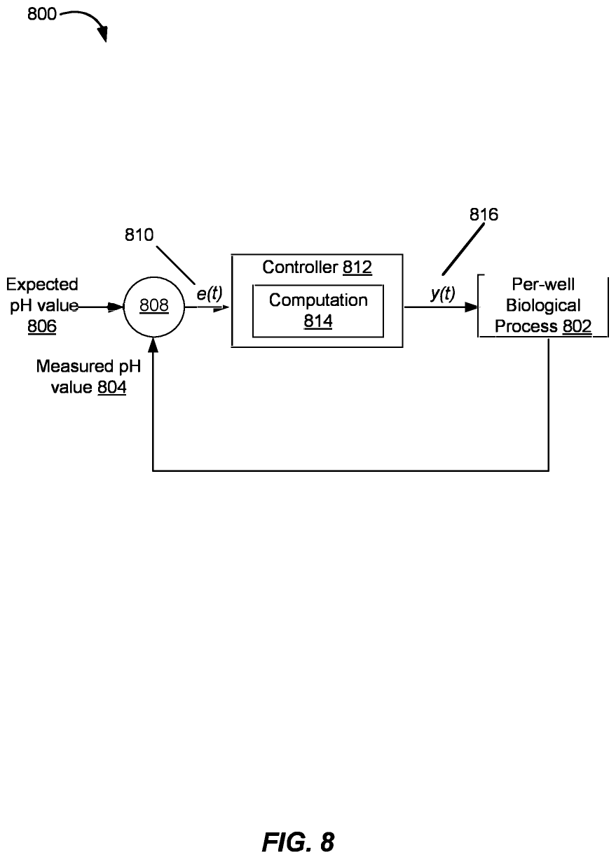

The pH measurement system 116 immediately transmits the measured pH value to a device having data processing capabilities, such as the controller 103 and/or computing device 104, for analysis. As part of the analysis and described in greater detail with respect to FIG. 8, pH control is initiated. For example, a measured pH value of the culture well 106 is compared to a predetermined pH value for the well, where the predetermined pH value can be obtained from the protocol. If the measured pH value deviates from the predetermined pH value, the pH adjustment system 118 is controlled by the controller 103 to cause an adjustment to the pH in the well to correct the deviation. In some examples, and as described in more detail with respect to FIG. 2 below, the pH adjustment system 118 can be a fluid system that enables a particular volume of pH adjusting fluid to be fed or dosed into the well in order to adjust the pH to correct the deviation (e.g., to conform with the predetermined pH value). This pH adjustment can occur in real-time once the measured pH value is obtained. For example, the pH adjustment can occur within about 10 seconds of obtaining the measurement.

As described above, the positioning device 114 successively moves or positions the pH measurement system 116 from well-to-well in a predefined measurement pattern or sequence every set interval of time. Therefore, in some examples, as one or more aspects of the pH control are performed (e.g., the comparison, deviation determination, and/or adjustment), the positioning device 114 can concurrently position the pH measurement system 116 in alignment with a next culture well 106 in the predefined measurement pattern, and the pH measurement system 116 can begin to measure the pH in the next culture well 106. In other words, the pH control is initiated before the pH measurement for the next culture well 106 is received, however, in some examples, the pH measurement for the next culture well 106 can be received as one or more aspects of the pH control are being performed. In further examples, the pH measurement for the next culture well 106 is not received until each of the aspects of the pH control are performed.

In addition to the one or more process parameters, such as pH, that are measured and automatically controlled by the system 100 other automated controls can be implemented by the instrument 102. Examples of other controls include automated temperature control, automated humidity control, automated feeding, automated gassing (e.g., oxygen and carbon dioxide), and automated revolutions per minute (RPM) (e.g., speed of movement performed by movement device 112).

FIG. 2 is a block diagram 200 of example components of the system 100 described in FIG. 1 that includes the instrument 102 and the computing device 104. The computing device 104 can execute an application 202 that is associated with (e.g., works in conjunction with) the instrument 102. For example, a user may utilize the application 202 executing on the computing device 104 to generate a protocol 204. As one illustrative example, the protocol 204 can be a cell culture protocol for an experiment associated with growth of a particular cell type.

As part of generating the protocol 204, the user can identify at least a subset of the culture wells 106 in the microplate 105 to be measured and one or more process parameters, such as pH, to be measured for each of those culture wells 106 over a plurality of cycles as the experiment is run. In some examples, each culture well 106 can have a same set of process parameters that are being measured. In other examples, at least one culture well 106 can have different process parameters that are being measured. Further, the particular process parameters that are being measured for a culture well 106 can be different between cycles, allowing for complex protocols to be generated.

In examples where the user selects for pH to be one of the process parameters measured per cycle, the user defines a pH profile comprising at least one expected pH value. The pH profile can be specific to each culture well 106 being measured. Additionally, the pH profile can be time-based causing the pH profile to include a plurality of predetermined pH values corresponding to different time periods or different cycles. As one illustrative example, for a first hour that the experiment is run, the predetermined pH value can be 7, for a second hour the predetermined pH value can be 6, and for a third hour the predetermined pH value can be 5, and so on. The user is also enabled to define a time interval between process parameter measurements per well. However, a lower limit of the time interval (e.g., a least amount of time between measurements) is fixed based on hardware constraints of the instrument 102.

Once the protocol 204 has been generated, the application 202 saves the protocol 204 locally to the computing device 104 or uploads the protocol 204 to an external storage system for remote storage (e.g., upload to a cloud-based storage system over a network such as the Internet), and transmits a copy of the protocol 204 to the controller 103 of the instrument 102. The controller 103 includes a memory 206 and a processing device 208. The controller 103 can store the copy of the protocol 204 in the memory 206. Then, when the instrument 102 is powered on, the controller 103 of the instrument 102 can retrieve a list of available protocols stored in memory 206, including the protocol 204, and provide the list for presentation via a display 210 of the instrument 102. The user can select the protocol 204 from the list for the instrument 102 and the controller 103 (e.g., based on instructions associated with the protocol 204 that are executed by the processing device 208) cause the instrument 102 to start an experiment corresponding to the protocol 204 (as well as later pause or stop the experiment). When the experiment is being run, the display 210 may present the selected protocol 204 and a representation of the process parameters that are being measured. Utilizing the display 210, the user can also change one or more a speed of the movement device 112, a temperature, and a gas concentration and gas flow rate (e.g., if gas supply is part of the protocol 204).

Additionally, the user can utilize the application 202 while an experiment is being run by the instrument 102 in accordance to the protocol 204 to modify the protocol 204 on the fly for one or more of the culture wells 106 for one or more future cycles. Example modifications can include modifications to pH profiles, timing between measurements, and selected process parameters among other examples. A copy of the modified protocol 204 can then be transmitted to the instrument 102 for storage in the memory 206 and presentation within the display 210.

For each cycle in which the pH is measured (e.g. alone or with other process parameters), the positioning device 114 is controlled by the controller 103 to successively move or position the pH measurement system 116 from well-to well in a predefined measurement pattern or sequence every set interval of time (e.g., as defined by the protocol 204) to enable at least pH in each culture well 106 to be measured.

As previously discussed in FIG. 1, the pH measurement system 116 can be an optical measurement system that includes an optical sensor device 212. The optical sensor device 212 can include an optical source 216 that emits electromagnetic radiation (e.g., light) to illuminate the culture well 106. Example types of the optical source 216 can include fiber optics or one or more light emitting diodes (LED), where the LEDs can be arranged in an array. In some examples, the optical sensor device 212 can include two or more optical sources 216 of same or differing types. Additionally, the optical sensor device 212 can include a sensor 218 that detects scattered light responsive to the emitted electromagnetic radiation from the optical source 216 and converts the detected light to electrical signals. An example sensor 218 (also referred to as a detector) includes a photodiode. Additionally, one or more filter modules can be used to control a specific range of wavelengths of light emitted by the optical source 216 and/or detected by the sensor 218.

Further, to facilitate pH measurement, the culture wells 106 each include at least one chemical sensor material, such as a fluorescent indicator solution (e.g., a fluorescent dye), that reacts specifically to environmental conditions in the culture well 106. For example, fluorescence indicator solutions have fluorescence characteristics that change in response to changes in pH value. This chemical sensor material can be immobilized in a polymer matrix, for example, on an inner, bottom surface of the culture well 106 causing the chemical sensor material to act as a pH optode 220. The light emitted from the optical source 216 can be emitted directly toward the pH optode 220, such that the scattered light detected by the sensor 218 and converted to the electrical signals includes fluorescence characteristics indicative of pH in the culture well 106. For example, a read out of the pH optode 220 can be performed by a time resolved measurement at specific wavelengths and a corresponding phase shift dependent on hydronium ions present can be used to calculate pH values. In some examples, the culture wells 106 can include more than the pH optode 220 depending on the number and types of process parameters to be measured. As one illustrative example, the culture wells 106 can also include a DO optode to facilitate DO measurement using this optical measurement system.

In some examples, a type of chemical sensor material is chosen based on a range of pH that the material sensitively (e.g., accurately) responds to, which further corresponds to a range of pH values within the pH profiles. The range of pH values within the pH profiles can be based on a particular cell type being cultured. For example, a range of pH values are typically narrower for mammalian cells than bacterial cells. The range can further be affected based on whether bacterial cells are anaerobic or aerobic, among other similar examples. An example range for pH measurements include a range of about pH 4 to pH 7.5. In other examples, the range may be extended from about pH 3.8 to pH 8.

For each cycle in which the pH is measured (e.g. alone or with other process parameters), the pH measurement system 116 can be controlled by the controller 103 to successively measure and record pH of each culture well 106 as the pH measurement system 116 is being positioned well-to-well by the positioning device 114.

As previously discussed in FIG. 1, the positioning device 114 can be an X-Y axes system. The X-Y axes system can be comprised of two perpendicular axes that move along rails using a motor and pulley mechanism. At the intersection point of the two axes, the optical source 216 of the optical sensor device 212 is fixed to both axes. Therefore, moving the axes pulls the optical source 216 underneath a culture well 106 of the microplate 105 to illuminate the culture well 106 and transport the scattered or the emitted light to the sensor 218 of the optical sensor device 212.

The optical sensor device 212 can control the emission of the electromagnetic radiation from the optical source 216 based on signals received from the controller 103. Additionally, the optical sensor device 212 can provide the electrical signals generated by the sensor 218 that are indicative of measured pH value, along with other measured process parameters if part of the protocol 204, to the controller 103 for analysis. In some examples, the electrical signals are provided to the controller 103 in real-time as the pH measurements are performed for each culture well 106. For example, when the pH of a culture well 106 is measured (e.g., a measured pH value is obtained), the signal is immediately provided to the controller 103. In some examples, the controller 103 can provide the measured pH value to the computing device 104, where the pH value can be displayed via a user interface provided by the application 202 on the computing device 104 to allow online monitoring.

When the controller 103 receives a measured pH value of a culture well 106, pH control is initiated by the controller 103. For example, the measured pH value is compared to the predetermined pH value in the culture well 106, where the predetermined pH value is obtained from the pH profile for the corresponding well included in the protocol 204. If the measured pH value deviates from the predetermined pH value, the pH in the well is adjusted to correct the deviation. In some examples, the pH control is initiated before a pH value of a next culture well 106 is measured by the pH adjustment system 118 and received by the application. For example, concurrent with the pH control, the positioning device 114 can position the optical sensor device 212 of the pH measurement system 116 in alignment with a next culture well 106 along the measurement pattern such that the optical sensor device 212 can then begin to measure pH in the next culture well 106. However, one or more of the comparison, the deviation determination, and pH adjustment is performed before the pH measurement is performed in the next culture well 106.

For the pH adjustment, the controller 103 transmits signals to the pH adjustment system 118 to cause adjustment of the pH in the culture well 106 to correct for the deviation. In some examples, the pH adjustment system 118 is a fluid system that includes a fluid source 222 (e.g., a reservoir) that contains pH adjusting fluid, and a fluidics device 223 for conveying the pH adjusting fluid from the fluid source 222 to each of the culture wells 106. In one example, the fluidics device 223 is comprised of a plurality of channels 224 from the fluid source 222 to each of the culture wells 106, and a plurality of valves 226 that open and close to control the movement or flow of the pH adjusting fluid from the fluid source 222 to a culture well 106 via a channel 224.

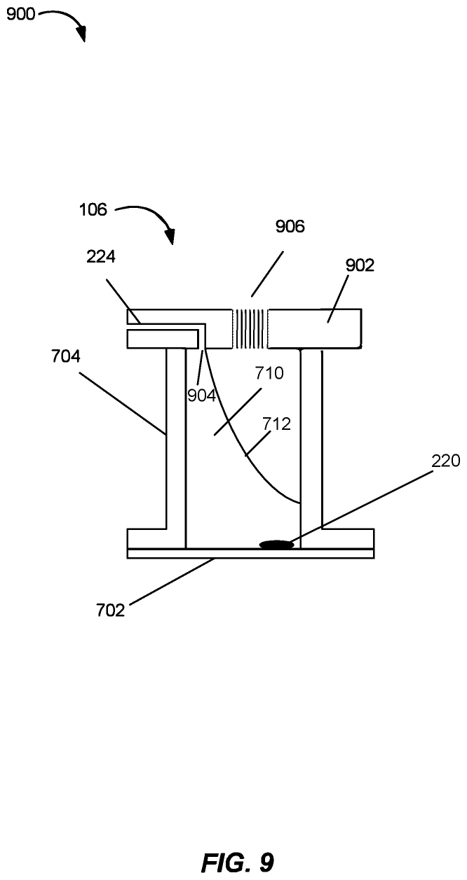

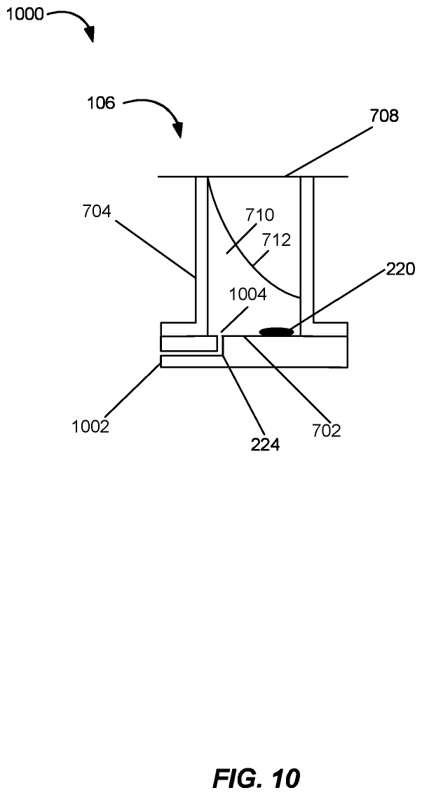

In some examples, the pH adjustment system 118 is at least partially integrated with the microplate 105 to enable the pH adjusting fluid to be dosed or fed into the culture wells 106 via the channels 224. For example, the channels 224 can be integrated with the microplate 105 (e.g., in a lid or cover of the microplate 105) such that they enter the culture wells 106 via an opening in a top surface of the culture wells 106, as shown in FIG. 9 below. In another example, the channels 224 can be integrated with the microplate 105 such that they enter the culture wells 106 via an opening in a bottom surface of the culture wells 106, as shown in FIG. 10 below. In a further example, the channels 224 can be integrated with the microplate 105 such that they enter the culture wells 106 via an opening in a sidewall surface of the culture wells 106.

In another example (not illustrated herein), the fluidics device 223 can be an automated dispensing system, such as a pipetting system or pipetting robot, that dispenses the pH adjusting fluid into the culture wells 106 (e.g., via a top surface of the culture wells 106).

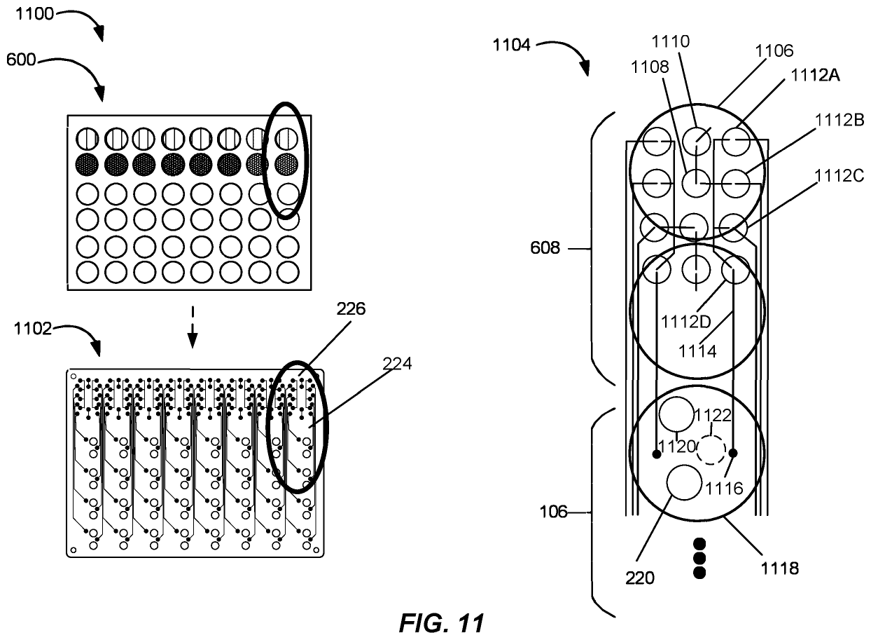

The fluid contained by the fluid source 222 can be in a liquid or a gas form. The fluid can be a pH adjusting fluid having properties that cause the pH in the culture well 106 to become more basic or more acidic. As illustrative, non-limiting examples, the fluid can include a liquid actuator or gas, such as sodium hydroxide (NaOH), hydrogen chloride (HCl), bicarbonate (HCO₃), or carbon dioxide (CO₂). In some examples, when the fluid is a liquid, reservoir wells of the microplate 105 can act as the fluid source 222, where the reservoir wells are positioned relative to a microfluidic chip housing the channels 224 and valves 226 that is integrated with the microplate 105, as shown in FIG. 11 below. In other examples, when the fluid is a gas, the pH adjustment system 118 can include a gas supply source as the fluid source 222 and a lid integrated with the microplate 105 can include structures to enable the gas to be supplied to each culture well.

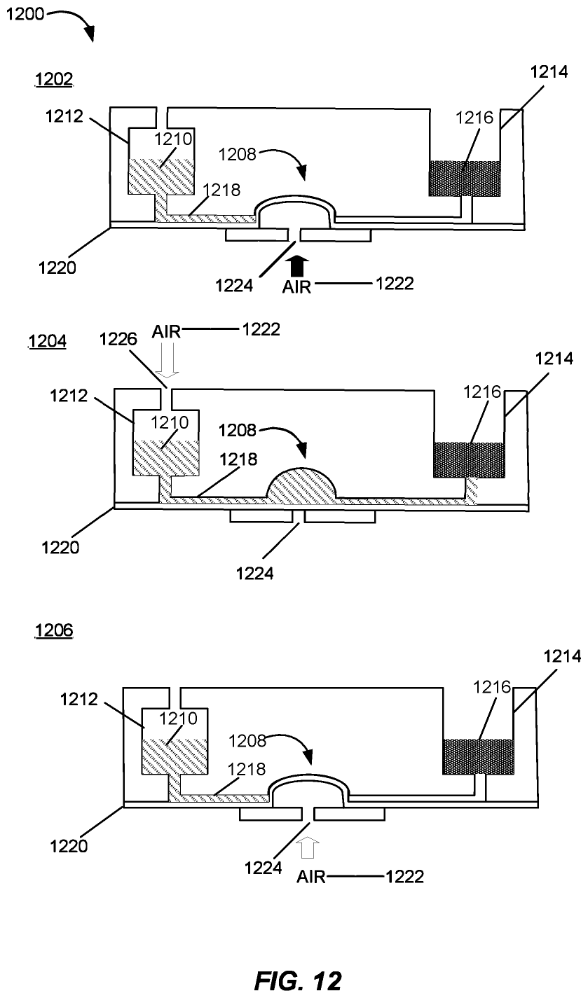

The valves 226 used to control fluid movement via the channels 224 can be pneumatically controlled via pneumatic components 228 of the instrument 102. For example, each pneumatic component 228 can be associated with a valve 226 and can receive compressed air from a compressed air source. As disclosed in greater detail with reference to FIG. 12 below, pressure applied by the compressed air source when activated can cause a valve 226 to close off or block a channel 224 to prevent fluid flow from the fluid source 222 to a culture well 106 via the channel 224. Therefore, the signal transmitted by the controller 103 to the pH adjustment system 118 to cause the pH to be adjusted in the culture well 106 can be a signal causing actuation of one or more of the pneumatic components 228 to cause one or more valves 226 to open to allow the pH adjusting fluid to flow from the fluid source 222 to the culture well 106 via a respective channel 224. An illustrative example is provided in FIG. 12 below. In some examples, the pneumatic components 228 can be integrated with the support 110 for the microplate 105, particularly when the channels 224 and valves 226 are positioned such that fluid enters the culture wells 106 via an opening the in the bottom surface of the culture wells 106 (e.g., if the channels 224 and valves 226 are contained within a microfluidic chip integrated with a bottom surface (e.g., positioned underneath) the microplate 105).

In some embodiments, the movement device 112 continues to shake the microplate 105 as the pH adjusting fluid is added to the culture well 106 via the channel 224. As a result of the motion provided by the movement device 112, the feed of oxygen into the fluid is improved, and thorough mixing of the fluids with the culture medium in the culture well 106 is achieved.

As one illustrative example, the addition of the pH adjusting fluid can occur within 10 seconds of the measurement of the pH value by the optical sensor device 212 of the pH measurement system 116. Resultantly, a volume of pH adjusting fluid that is added to the culture well 106 to correct for the deviation of the measured pH value from the predetermined pH value for the culture well 106 is highly accurate (e.g., before the pH value fluctuates) to cause the desired pH adjustment within the culture well 106.

Additionally, in some examples, as the pH is measured and any pH adjustments to the culture well 106 are made, the controller 103 can provide associated measurement and adjustment information to the application 202 via a user interface provided by the application 202 on the computing device 104 to allow online monitoring.

As described with reference to FIG. 2, the controller 103 of the instrument 102 can form a closed control loop with the pH measurement system 116 and the pH adjustment system 118 to receive measured pH values from the pH measurement system 116, determine whether pH adjustment is needed to correct for a deviation from predetermined values, and, if so, cause the pH adjustment system 118 to adjust the pH to correct the deviation. Additionally, the controller 103 can provide the signals or instructions to control the various components of the instrument 102, including the movement device 112, the positioning device 114, the pH measurement system 116, the pH adjustment system 118 and the pneumatic components 228. In other examples, the application 202 executing on the computing device 104 can form the closed control loop with the pH measurement system 116 and the pH adjustment system 118 and/or provide the signals or instructions to control the various components of the instrument 102. In such examples, the signals or instructions can be provided directly to those components from the application 202 or they may be provided indirectly via the controller 103.

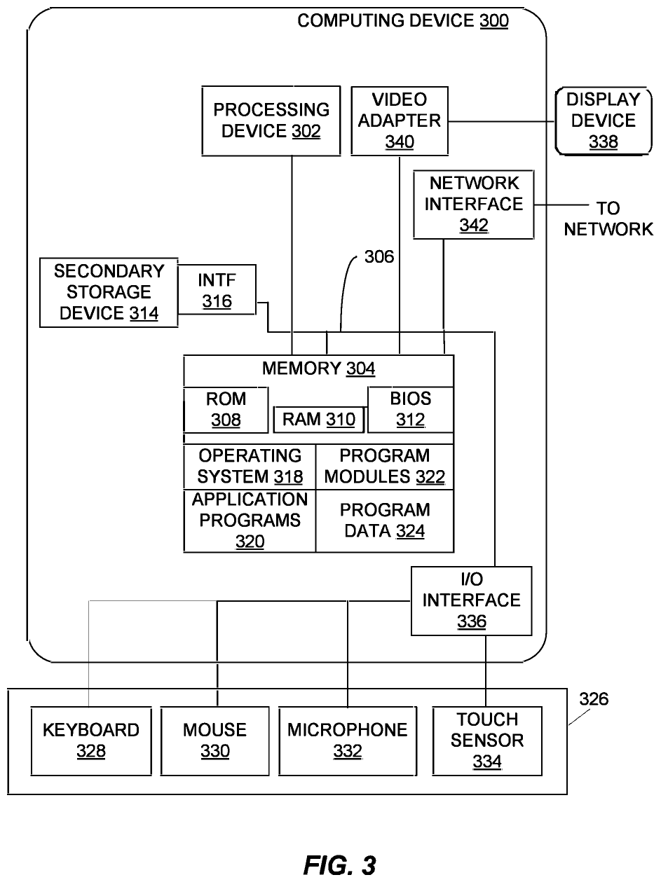

FIG. 3 is an example computing device 300 that can be used to implement aspects of the present disclosure. The computing device 300, for example, may provide an operating environment for controller 103 and computing device 104. The computing device 300 can be used to execute the operating system, application programs, and software modules (including the software engines) described herein.

The computing device 300 includes, in some embodiments, at least one processing device 302, such as a central processing unit (CPU). A variety of processing devices are available from a variety of manufacturers, for example, Intel or Advanced Micro Devices. In this example, the computing device 300 also includes a system memory 304, and a system bus 306 that couples various system components including the system memory 304 to the processing device 302. The system bus 306 is one of any number of types of bus structures including a memory bus, or memory controller: a peripheral bus; and a local bus using any of a variety of bus architectures.

The system memory 304 includes read only memory (ROM) 308 and random access memory (RAM) 310. A basic input/output system (BIOS) 312 containing the basic routines that act to transfer information within computing device 300, such as during start up, is typically stored in the ROM 308.

The computing device 300 also includes a secondary storage device 314 in some embodiments, such as a hard disk drive, for storing digital data. The secondary storage device 314 is connected to the system bus 306 by a secondary storage interface 316. The secondary storage device 314 and their associated computer readable media provide nonvolatile and non-transitory storage of computer readable instructions (including application programs and program modules), data structures, and other data for the computing device 300.

Although the exemplary environment described herein employs a hard disk drive as a secondary storage device, other types of computer readable storage media are used in other embodiments. Examples of these other types of computer readable storage media include flash memory cards, digital video disks, compact disc read only memories, digital versatile disk read only memories, random access memories, or read only memories. Some embodiments include non-transitory media. Additionally, such computer readable storage media can include local storage or cloud-based storage.

A number of program modules can be stored in secondary storage device 314 or system memory 304, including an operating system 318, one or more application programs 320, other program modules 322 (such as the software engines described herein), and program data 324. One example application program includes an application executed by the controller 103 of the instrument 102 to, among other things, provide automated pH control. Another example application program includes the application 202 that is executing on the computing device 104 and is associated with (e.g., works in conjunction with) the instrument 102 to, among other things, generate and/or modify protocols for experiments to be run by the instrument 102 and enable online monitoring of various process parameters, such as pH, defined by the protocols. In some embodiments, the application 202 can also send instructions to the controller 103 to provide or facilitate automated pH control. The computing device 300 can utilize any suitable operating system, such as Microsoft Windows™, Google Chrome™ OS, Apple OS, Unix, or Linux and variants and any other operating system suitable for a computing device. Other examples can include Microsoft, Google, or Apple operating systems, or any other suitable operating system.

In some embodiments, a user provides inputs to the computing device 300 through one or more input devices 326. Examples of input devices 326 include a keyboard 328, mouse 330, microphone 332, and touch sensor 334 (such as a touchpad or touch sensitive display). Other embodiments include other input devices 326. The input devices are often connected to the processing device 302 through an input/output interface 336 that is coupled to the system bus 306. These input devices 326 can be connected by any number of input/output interfaces, such as a parallel port, serial port, game port, or a universal serial bus. Wireless communication between input devices and the input/output interface 336 is possible as well, and includes infrared, BLUETOOTH® wireless technology, IEEE 802.11a/b/g/n, cellular, ultra-wideband (UWB), ZigBee, LoRa, or other radio frequency communication systems in some possible embodiments.

In this example embodiment, a display device 338, such as a monitor, liquid crystal display device, projector, or touch sensitive display device, is also connected to the system bus 306 via an interface, such as a video adapter 340. In addition to the display device 338, the computing device 300 can include various other peripheral devices (not shown), such as speakers or a printer.

When used in a local area networking environment or a wide area networking environment (such as the Internet), the computing device 300 is typically connected to the network through a network interface 342, such as an Ethernet interface. Other possible embodiments use other communication devices. For example, some embodiments of the computing device 300 include a modem for communicating across the network.

The computing device 300 typically includes at least some form of computer readable media. Computer readable media includes any available media that can be accessed by the computing device 300. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 104.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device 300 illustrated in FIG. 3 is also an example of programmable electronics, which may include one or more such computing devices, and when multiple computing devices are included, such computing devices can be coupled together with a suitable data communication network so as to collectively perform the various functions, methods, or operations disclosed herein.

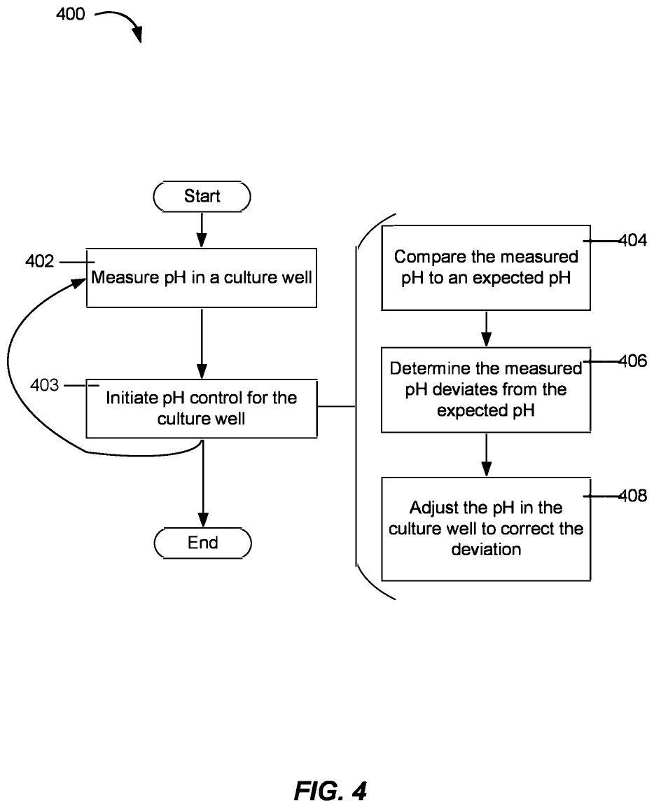

FIG. 4 is a flowchart illustrating a method 400 for controlling pH. The method 400 can be performed by a biological process control system, such as the system 100 described with reference to FIG. 1 and FIG. 2, to adjust pH of individual culture wells 106 in a microplate 105 in real-time as measured pH values for the culture wells 106 are obtained in accordance with a protocol, such as the protocol 204. Method 400 can be implemented as part of a closed control loop that is driven by the controller 103 of the instrument 102.

The method 400 begins at operation 402, where pH in a culture well 106 is measured. The pH can be measured using the pH measurement system 116. For example, the positioning device 114, controlled by the controller 103, positions the optical sensor device 212 in alignment with the culture well 106 and particularly with the pH optode 220 in the culture well 106. The optical sensor device 212, controlled by the controller 103, then causes the optical source 216 of the optical sensor device 212 to emit electromagnetic radiation (e.g., light) into the culture well 106. The sensor 218 of the optical sensor device 212 detects scattered light responsive to the emitted electromagnetic radiation and converts the detected light to one or more electrical signals, where the detected light can include fluorescence characteristics of the pH optode 220 that is indicative of pH in the culture well 106. The optical sensor device 212 provides the electrical signals to the controller 103 for processing.

The method 400 can proceed to operation 403 to initiate pH control, where pH control can be comprised of operations 404, 406, and 408. For example, the measured pH in the culture well 106 is compared to a predetermined pH in the culture well 106 at operation 404. For example, the controller 103 receives, from the pH measurement system 116, a signal including the measured pH value. Additionally, the controller 103 can retrieve the predetermined pH value from a pH profile specific to the culture well 106 that was generated as part of the protocol 204. This expected pH value is then compared to the measured pH value.

At operation 406 of the pH control, the measured pH is determined to deviate from the predetermined pH based on the comparison. The deviation or difference between the measured pH and the predetermined pH can also be referred to as an error. The deviation can include both a direction (e.g., more acidic than predetermined pH or more basic than predetermined pH) and a magnitude (e.g., a pH value difference).

In response to determining the deviation at operation 406, the pH in the culture well 106 can be adjusted to correct the deviation at operation 408 of the pH control. For example, a particular volume of pH adjusting fluid to cause the pH in the culture well 106 to become equal to or within at least a predefined range of the predetermined pH is determined. In some examples, the pH is adjusted only if the deviation meets a threshold value.

The pH adjustment system 118, controlled by the controller 103, can then feed or dose the culture well 106 with the particular volume of pH adjusting fluid. As one example, the application 202 can control the opening of one or more valves 226 that are currently closing off or blocking one or more channels 224 between the fluid source 222 and the culture well 106 in order to allow the pH adjusting fluid to flow from the fluid source 222 to the well via the channels 224. In some examples, such control includes deactivating a pneumatic component 228 of a respective valve 226 such that compressed air is no longer being received and the associated pressure applied.

The pH adjustment for the culture well 106 can occur in real-time following the pH measurement. As one illustrative example, the pH adjustment in the culture well 106 can occur within about 10 seconds of the pH measurement in the culture well 106. Resultantly, the volume of pH adjusting fluid that is added to the culture well 106 to correct for the deviation of the measured pH from the predetermined pH for the culture well 106 is highly accurate (e.g., occurring before the pH fluctuates).

In some examples, the method 400 may proceed from operation 402 to operation 403 prior to a next culture well being measured. For example, at least partial performance of operation 404 may occur before the pH measurement for the next culture well 106 is received. In other examples, one or more of operations 404, 406 and 408 may have been performed or at least partially performed before the pH is measured for the next culture well 106. As an illustrative example, following pH measurement of the culture well 106, the positioning device 114, controlled by the controller 103, positions the optical sensor device 212 in alignment with a next culture well 106 in the predefined measurement pattern and begins to perform method 400 with respect to the next culture well 106 at operation 402 by measuring pH in the next culture well 106. However, in this example, the pH measurement of that next culture well 106 may not be performed until at least operation 404 has been partially performed. In some examples, the time interval by which the optical sensor device 212 of the pH measurement system is moved well-to-well is defined in the protocol 204 and can be based on a number and type of process parameters, if any, that are being measured in addition to pH.

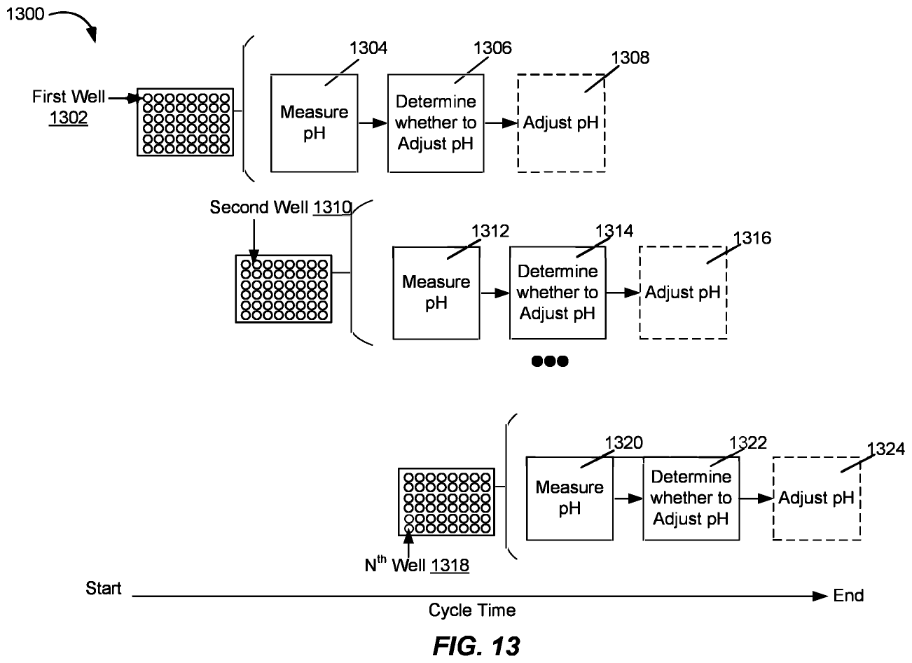

Per cycle, the method 400 can be performed for each culture well 106 that is identified for pH measurement within the protocol 204. Additionally, as indicated above, once pH has been measured in a culture well 106, the optical sensor device 212 can be positioned with respect to a next culture well 106 to measure pH in the next culture well 106. Thus, in some examples at least some operations of method 400 may overlap for one or more culture wells 106 as conceptually illustrated in FIG. 13 below. For example, once pH control is initiated for one culture well 106 (e.g., at least operation 404 has been partially performed), pH can be measured for another.

As described with reference to FIG. 4, the controller 103 of the instrument 102 may drive the closed control loop and control various components of the instrument 102, including the positioning device 114, the pH measurement system 116, and the pH adjustment system 118, among other components, to enable the automated pH control provided by method 400. Alternatively, in other embodiments, the application 202 executing on the computing device 104 can drive the closed control loop and/or control the various components of the instrument 102 either directly or via the controller 103.

FIG. 5A is an example microplate configuration and associated pH measurement pattern. FIG. 5B is another exemplary pH measurement pattern for the microplate configuration shown in FIG. 5A. Referring concurrently to FIGS. 5A and 5B, microplate 500 is one example type of microplate 105 that can be used with the instrument 102. The microplate 500 includes a plurality of culture wells 106 arranged in rows 502 and columns 504. As illustrated in this example, the microplate 500 includes 48 culture wells arranged in 6 rows and 8 columns. The culture wells 106 contain culture medium that has been inoculated with microorganisms and incubated, where biological processes occurring within the culture wells 106 and the effects thereof are monitored and controlled by the system 100, including pH fluctuations. Additionally, each of the culture wells or at least each of a plurality of the culture wells 106 can include at least one optode, such as a pH optode 220 described with reference to FIG. 2, to facilitate pH measurement in the culture well 106.

If all culture wells 106 have been selected to be measured via a protocol (e.g., such as protocol 204 described with reference to FIG. 2), then the pH measurement system 116 of system 100 can measure each of the culture wells 106 in a predefined measurement pattern. As illustrated in FIG. 5A, one exemplary predefined measurement pattern 506 can be a serpentine pattern that begins with a first culture well 106 at a top left of the culture wells 106, proceeds horizontally in a first direction along a first row of the culture wells 106, measuring each culture well 106 along the first row successively. Following measurement of the culture wells 106 in the first row, the pH measurement system 116 is moved downward to a second row of the culture wells 106. The predefined measurement pattern 506 then proceeds horizontally in a second direction (e.g., opposite to the first direction) along the second row of the culture wells 106 and downward to a third row of the culture wells 106 and so on, until the pattern ends with an Mh culture well 106 at the bottom left of the culture wells 106. In other embodiments, and as shown in FIG. 5B, a raster scan pattern 508 can be employed to measure the culture wells 106 of the microplate 500, where the pH measurement system 116, after measuring the last column of each row, is moved to the first column of the next row. Although two particular patterns are presented as examples herein, any suitable pattern may be employed.

Referring back to the microplate 500 described in FIGS. 5A and 5B, in some examples, when the microplate 500 is implemented, at least the fluid source 222 of the pH adjustment system 118 is separate from (e.g., not integrated with) the microplate 500. For example, fluid from the fluid source 222 can be automatically fed to the culture wells from the top through a lid or cover of the microplate 500 that comprises the channels 224 as shown in FIG. 9 or from the bottom via a plane underneath the microplate 500 that comprises the channels 224 as shown in FIG. 10. In some examples, the fluid can be pipetted from the top into the culture wells using an automated dispensing system (e.g., as part of an autosampling system).

FIG. 6 is another example microplate configuration and associated pH measurement pattern. Microplate 600 is one example type of microplate 105 that can be used with the instrument 102. The microplate 600 includes a plurality of wells 602 of two different types that are arranged in rows 604 and columns 606. The first type of the plurality of wells 602 include reservoir wells 608 and the second type of the plurality of wells 602 include the culture wells 106. The reservoir wells 608 can contain liquid reagents that are automatically fed into culture wells 106. For example, the reservoir wells 608 include the fluid source 222 of the pH adjustment system 118. As illustrated in this example, the microplate 600 includes 48 wells arranged in 6 rows and 8 columns, where the upper two rows are the reservoir wells 608 and the bottom four rows are the culture wells 106 to yield 16 reservoir wells 608 and 32 culture wells 106.

As described in more detail below with reference to FIG. 11, each reservoir well 608 can feed the reagents to a subset of the culture wells 106 that are arranged in a same column 606 of the microplate 600 as the reservoir well 608. In some examples and as illustrated in FIG. 6, a first subset 612 (e.g., first row) of the reservoir wells 608 and a second subset 614 (e.g., second row) of the reservoir wells 608 contain different reagents. In one example, the first subset 612 of the reservoir wells 608 can contain nutrient solution that promotes microbial growth, for example, and the second subset 614 of the reservoir wells 608 can contain pH adjusting solution used by the pH adjustment system 118 to adjust pH. In another example, the first subset 612 and the second subset 614 of the reservoir wells 608 contain pH adjusting solution of different types. For example, the pH adjusting solution in the first subset 612 can be acidic and the pH adjusting solution in the second subset 614 can be basic or vice versa.

Similar to the culture wells discussed with reference to FIGS. 5A and 5B, the culture wells 106 in the example illustrated in FIG. 6 contain culture medium that has been inoculated with microorganisms and incubated, where biological processes occurring within the culture wells 106 and the effects thereof are monitored and controlled by the system 100, including pH fluctuations. Additionally, each of the culture wells 106 can include at least one optode, such as a pH optode 220 described with reference to FIG. 2, to facilitate pH measurement in the culture well 106.

If all culture wells 106 have been selected to be measured via a protocol (e.g., such as protocol 204 described with reference to FIG. 2), then the pH measurement system 116 of system 100 can measure each of the culture wells 106 in a predefined measurement pattern 616. As illustrated in FIG. 6, an exemplary predefined measurement pattern 616 begins with a first culture well 106 at a top left of the culture wells 106, proceeds horizontally in a first direction along a first row of the culture wells 106 and downward to a second row of the culture wells 106. The predefined measurement pattern 616 then proceeds horizontally in a second direction (e.g., opposite to the first direction) along the second row of the culture wells 106 and downward to a third row of the culture wells 106 and so on, until the pattern ends with an N^{th} culture well 106 at the bottom left of the culture wells 106. As described above with respect to FIGS. 5A and 5B, any suitable pattern may be employed.

FIG. 7 is a conceptual diagram 700 illustrating a pH measurement of an individual culture well 106. As illustrated, the culture well 106 can be bounded by a circular and/or cylindrical bottom surface 702 that is permeable to electromagnetic radiation (e.g., light) and a cylindrical casing comprising a side wall 704. In some examples, the culture well 106 can further be bounded by a top surface 708, such as a lid, a cover, or other similar seal. In other examples, the culture well 106 can have a cross-section intersecting the side wall 704 parallel to the bottom surface 702, the cross-section having a shape diverging from a round, cylindrical, square or rectangular shape.

The culture well 106 can contain culture medium 710 that has been inoculated with microorganisms and incubated. In some examples, the culture well 106 is continuously moved (e.g., shaken or rotated about an axis) by the movement device 112, causing a fluid crest 712 to form in the culture medium 710. Additionally, the culture well 106 can include a pH optode 220. The pH optode 220 is a chemical sensor material such as a fluorescent indicator solution or fluorescent dye having fluorescence characteristics that change in response to pH changes in the culture well 106. The pH optode 220 can be immobilized on the bottom surface 702 of the culture well 106.

In some examples, the optical sensor device 212 of the pH measurement system 116 is aligned under the culture well 106 in such a manner that the electromagnetic radiation is emitted at a wavelength between about 200 nanometers (nm) and 25 micrometers (μm), directly and exclusively into the culture well 106 in the form of a light beam 714 to record a measured value for pH in the culture well 106. For example, the light beam 714 emitted from the optical sensor device 212 can be emitted directly toward the pH optode 220 in the culture well 106 such that the scattered light 716 detected by the optical sensor device 212 responsive to the emitted light beam 714 includes fluorescence characteristics indicative of pH in the culture well 106. In some examples, one or more additional process parameters such as DO, biomass, and fluorescence intensity of fluorescing molecules or proteins can be measured utilizing the optical sensor device 212.

As previously discussed, in some examples, the culture well 106 continues to be moved (e.g., shaken orbitally, shaken linearly, rotated about an axis that extends through a point on the culture well 106, rocked in a reciprocating manner) by the movement device 112 as the process parameters are measured. In examples where the movement device 112 is an orbital shaker, a diameter of an associated orbit by which the culture well 106 is orbitally shaken can be chosen to be less than or equal to a diameter of the bottom surface 702 of the culture well 106 such that the light beam 714 emitted from the optical sensor device 212 remains directed to only one well (e.g., the particular well underneath which the optical sensor device 212 is positioned).

Further examples for measuring process parameters are described in U.S. Pat. No. 8,268,632 issued on Sep. 18, 2012, and titled METHOD AND DEVICE FOR RECORDING PROCESS PARAMETERS OF REACTION FLUIDS IN SEVERAL AGITATED MICROREACTORS, the disclosure of which is hereby incorporated by reference in its entirety for all purposes and specifically for description of measuring process parameters, such as pH, of an individual culture well.

FIG. 8 is a conceptual diagram illustrating a closed control loop 800 implemented by the system 100 for pH control. The closed control loop 800 measures, evaluates, and controls one or more variables of a process such as the biological processes occurring in individual culture wells 106 of the microplate (e.g., a per-well biological process 802). In this example, the variable of the per-well biological process 802 of interest is pH.

In some embodiments and as discussed with reference to FIG. 8, the controller 103 of the instrument 102 drives the closed control loop 800. In alternative embodiments, the application 202 executing on the computing device 104 can drive the closed control loop 800. In one example, as part of the closed control loop 800, the controller 103 successively receives a measured pH value 804 for each culture well 106 from the pH measurement system 116 as it is being moved well-to-well via the positioning device 114 to measure pH of each culture well 106. The controller 103 retrieves a predetermined pH value 806 from the pH profile for the corresponding culture well 106 included in the protocol 204.

At a first operation 808, the controller 103 determines a difference (e(t)) 810 between the predetermined pH value 806 and the measured pH value 804. If a difference is determined (e.g., there is a deviation or an error), then a controller 812 of the closed control loop 800 (e.g., executed by the controller 103) performs a computation 814 to determine a correcting variable (y(t)) 816 to correct the deviation. Based on the correcting variable (v(t)) 816, a volume of pH adjusting fluid to be added to the respective culture well 106 is determined and the pH adjustment system 118 acts as a control element of the closed control loop 800 to dose the culture well 106 with that volume of pH adjusting fluid to change or influence the biological process 802 for that culture well 106 in order to correct the deviation or error from the predetermined pH value 806. In some examples, pH is adjusted only when the determined difference exceeds a predetermined threshold value. For example, the controller 812 may only perform the computation 814 and determine the correcting variable (y(t)) 816 when such a threshold value is exceeded. Alternatively, the correcting variable (y(t)) 816 may be determined, but the pH adjustment system 118 may not act.

In one example, the controller 812 is a proportional-integral (PI) controller (or a proportional-integral-derivative controller) that determines a correction for the deviation based on a proportional (P) component and an integral (I) component. In such examples, the computation 814 performed to determine the correcting variable (y(t)) is as follows:

$$y ( t ) = \frac{e ( t )}{{K}_{P}} \cdot {K}_{I} \cdot {\int }_{0}^{t} e ( t ) d τ$$

where y(t) 816 is the correcting variable, e(t) 810 is the difference between the predetermined pH value 806 and the measured pH value 804, Kₚ is a proportional factor of P component, and Kris a proportional factor of I component.

The P component describes a linear dependency between the measured pH value 804 and the correcting variable y(t) 816. Therefore, the larger the value of the deviation or error (e.g., the larger the value of e(t) 810), the larger the correcting variable y(t) 816. In general, lowering the P component or increasing a time period for which a respective valve 226 of the pH adjustment system 118 will be open to allow a larger dosing of pH adjusting fluid into the culture well 106 via a respective channel 224 can result in a faster and stronger response to correct the deviation.

FIG. 9 illustrates an example configuration 900 for components of the pH adjustment system 118 to enable pH adjustment of an individual culture well 106. Similar to the individual culture well 106 described with reference to FIG. 7, the culture well 106 of FIG. 9 can be bounded by a circular and/or cylindrical bottom surface 702 that is permeable to light, a cylindrical casing comprising a side wall 704, and a top surface, such as a cover 902 in the configuration 900. In other examples, the culture well 106 can have a cross-section intersecting the side wall 704 parallel to the bottom surface 702, the cross-section having a shape diverging from a round, cylindrical, square or rectangular shape. Examples of these type of culture wells that have cross-sections with diverging shapes are described in U.S. Pat. No. 8,828,337 issued on Sep. 9, 2014, and titled MICROREACTOR, the disclosure of which is hereby incorporated by reference in its entirety for all purposes and specifically for description of culture wells having cross-sections with diverging shapes.

The culture well 106 can contain culture medium 710 that has been inoculated with microorganisms and incubated, where continuous movement of the culture well 106 by the movement device 112 causes a fluid crest 712 to form in the culture medium 710. Additionally, the culture well 106 can include a pH optode 220 immobilized on the bottom surface 702 of the culture well 106 to facilitate the pH measurement.

As shown in configuration 900 the cover 902 includes the channels 224 of the pH adjustment system 118 that allows pH adjusting fluid from the fluid source 222 to be fed or dosed into each of the individual culture wells 106 through an opening 904 in the cover 902. To ensure that a fluid droplet presented by the channel 224 via the opening 904 in the cover 902 can also be taken up in small quantities by the culture medium 710, the fluid crest 712 generated by the movement (e.g., shaking) provided by the movement device 112 extends to meet the opening 904. In some examples, the cover 902 can also comprise aeration inlets 906 with membrane inserts or other similar structures to ensure sterile gas supply into the culture well 106 if required for the protocol 204.

In other examples (not illustrated herein), the pH adjusting fluid can be fed or dosed into the culture well 106 through a top surface such as the cover 902 via a pipette through one or more apertures in the cover 902 using an automated dispensing system (e.g., as part of an autosampler system).

FIG. 10 illustrates another example configuration 1000 for components of the pH adjustment system 118 to enable pH adjustment of an individual culture well 106. Similar to the individual culture well 106 described with reference to FIG. 7, the culture well 106 of FIG. 10 can be bounded by a circular and/or cylindrical bottom surface 702 that is permeable to light, a cylindrical casing comprising a side wall 704, and a top surface 708. In some examples, the top surface 708 is a gas-permeable membrane to ensure sterile gas infeed to the culture well 106. In other examples, the culture well 106 can have a cross-section intersecting the side wall 704 parallel to the bottom surface 702, the cross-section having a shape diverging from a round, cylindrical, square or rectangular shape.

The culture well 106 can contain culture medium 710 that has been inoculated with microorganisms and incubated, where continuous movement of the culture well 106 by the movement device 112 causes a fluid crest 712 to form in the culture medium 710. Additionally, the culture well 106 can include a pH optode 220 immobilized on the bottom surface 702 of the culture well 106 to facilitate the pH measurement.

In this configuration 1000, a plane 1002 underneath the bottom surface 702 of the culture well 106 includes the channels 224 through which the fluids are conveyed from the fluid source 222 to the individual culture wells 106 via an opening 1004 in the bottom surface 702 of the culture well 106. In one example, the plane 1002 can be a microfluidic chip integrated with a bottom surface of the microplate 105 containing the culture well 106, as described in detail with reference to FIG. 11 below: Similar to the bottom surface 702 of the culture well 106, the plane 1002 can be permeable to electromagnetic radiation to prevent any interference with the light from the optical sensor device 212 reaching the pH optode 220 during pH measurement. The opening 1004 can also be positioned on the bottom surface 702 of the culture well 106 to avoid any interference with the pH optode 220 (or any other optodes if present). In some examples, droplets of fluid presented through the channel 224 via the opening 1004 in the bottom surface 702 of the culture well 106 are taken up into the culture medium 710 by the fluid crest 712 generated by the movement (e.g., shaking) provided by the movement device 112.

FIG. 11 illustrates an example pH adjustment system 118 that is integrated with a microplate 105 (e.g., integrated system 1100). The integrated system 1100 can include a microfluidic chip 1102 that is positioned underneath a bottom surface of a microplate that includes a combination of reservoir wells and culture wells, such as microplate 600 described with reference to FIG. 6. A magnified view 1104 of a portion of the microplate 600 integrated with the microfluidic chip 1102, including two reservoir wells 608 and one of the culture wells 106 in a column 606 of the microplate 600 is illustrated in FIG. 11. As previously discussed, in some examples, each of the reservoir wells 608 in the column 606 contain reagents utilized for different purposes, such as a nutrient solution that promotes microbial growth in one (e.g., a feeding solution) and a pH adjusting fluid in the other (e.g., a pH solution). In other examples, both of the reservoir wells 608 in the column can contain pH adjusting solution, where one can contain acidic pH adjusting solution and the other can contain basic pH adjusting solution.

The microfluidic chip 1102 can be part of the pH adjustment system 118 housing the channels 224 and valves 226, where the valves 226 can be positioned on an edge of the microfluidic chip 1102. In some examples, the microfluidic chip 1102 is integrated with (e.g., positioned underneath) a bottom surface of the microplate 600 such that the edge comprising the valves 226 of the microfluidic chip 1102 is positioned directly below the reservoir wells 608 of the microplate 600 that serve as the fluid source 222 of the pH adjustment system 118. For example, a subset of the valves 226 correspond to each reservoir well 608. Additionally, placement of the valves 226 on the edge of the microfluidic chip 1102 preserves an optical area (e.g., an area permeable to electromagnetic radiation) below the microplate 600 and bottom surfaces of the wells 602 therein to enable optical measurement of process parameters, such as pH, dissolved oxygen, biomass, and fluorescence intensity of fluorescing molecules or proteins.

Turning to the magnified view 1104, an example subset of the valves 226 for a reservoir well 608, such as reservoir well 1106, include an inlet valve 1108, a pump valve 1110 and a plurality of outlet valves 1112A, 1112B, 1112C, 1112D (collectively, outlet valves 1112). Using the pump valve 1110, fluid from the reservoir well 1106 is taken up through the inlet valve 1108. The pump valve 1110 can define a volume of fluid that is taken is up. In some examples, the inlet valve can be attached to a chamber (not shown here) that temporarily contains the fluid, where the fluid builds pressure in the chamber once the inlet valve is closed and until an outlet valve 1112 is opened. In some examples, one or more of the inlet valve 1008, the pump valve 1110, and outlet valves 1112 are pneumatically controlled (e.g., by pneumatic components 228 in response to signals received from the controller 103 described with reference to FIG. 2.). An example of the pneumatic control mechanism is included in FIG. 12 below.

The outlet valves 1112 lead to channels 224 that direct fluids to outlets of respective culture wells 106 in the column 606. As one example, outlet valve 1112D leads into channel 1114 that directs fluids to an outlet 1116 of a culture well 1118 of the culture wells 106 in the column 606. As illustrated here, the subset of the valves 226 for the reservoir well 1106 include four outlet valves 1112 allowing the reservoir well 1106 to supply fluid to four culture wells 106 (e.g., the four culture wells 106 including the culture well 1118 that are in the same column 606 as the reservoir well 1106 in the microplate 600). As illustrated, the culture well 1118 can include a pH optode 220 for measuring pH as well as at least one other optode for measuring DO (e.g . . . , DO optode 1120) and capabilities to measure biomass or fluorescence intensity of fluorescing molecules or proteins (e.g., represented by element 1122). As previously discussed, placement of the valves 226 on the edge of the microfluidic chip 1102 preserves an optical area below the microplate 600 and bottom surface of the culture well 1118 to enable optical measurement of these process parameters.

Further examples of microfluidic chips that are similarly integrated with microplates are described in U.S. Pat. No. 10,421,071 issued on Sep. 24, 2019, and titled MICROREACTOR SYSTEM, the disclosure of which is hereby incorporated by reference in its entirety for all purposes and specifically for description of the microfluidic chip and a manner in which it can be integrated with a microplate.

FIG. 12 is a conceptual diagram 1200 illustrating pneumatically controlled valves of an example pH adjustment system 118. Configuration 1202, 1204 and 1206 illustrate an example valve 1208 that controls movement of fluid 1210 from a reservoir well 1212 to a culture well 1214 containing culture medium 1216 via a channel 1218. The culture well 1214 can be a culture well 106 of microplate 500 or a culture well 106 of microplate 600, among other examples.

A thin membrane 1220 is positioned underneath the channel 1218 in a pneumatically actuated control plane. As one example, the pneumatically actuated control plane and a fluid plane comprising the channel 1218, such as plane 1002 described with reference to FIG. 10 or the microfluidic chip 1102 described with reference to FIG. 11, are integrated with (e.g., form a part of) a bottom surface of a microplate containing the culture well 1214.

As shown in a first configuration 1202, the membrane 1220 can be pressurized with compressed air 1222 through a pneumatic duct 1224 causing the thin membrane 1220 to push into the channel 1218 and essentially close off the channel 1218 (e.g., forming a closed valve configuration). As a result, the fluid 1210 is unable to flow from the reservoir well 1212 to the culture well 1214. The pneumatic duct 1224 is one example pneumatic component 228 described with reference to FIG. 2.

When the compressed air 1222 is no longer being provided through the pneumatic duct 1124 and the pneumatic pressure over the membrane 1220 is sufficiently small compared to a pressure in the fluid channel 1218, the valve 1208 will open as shown in second configuration 1204. Achieving this pressure difference can be facilitated by providing compressed air 1222 to a pump valve 1226 (e.g., similar to pump valve 1110 discussed with reference to FIG. 11) associated with the reservoir well 1212. A pneumatic duct providing the compressed air 1222 to the pump valve 1226 (not shown here) is another example pneumatic component 228 described with reference to FIG. 2. When the valve 1208 opens, the fluid 1210 flows from the reservoir well 120 via the channel 1218 to the culture well 1214 for mixing with the culture medium 1216. In some examples, the fluid 1210 is a pH adjusting fluid that is mixed with the culture medium 1216 to adjust a pH of the culture medium 1216 in response to a determination that a measured pH value deviates from a predetermined pH value.

The valve 1208 closes again when the membrane 1220 is pressurized with the compressed air 1222 through a pneumatic duct 1224, as shown in a third configuration 1206.

A supply for the compressed air 1222 can be externally connected to the pneumatic components 228 described with reference to FIG. 2. In some examples, the pneumatic components 228 are provided in the support 110 of the movement device 112 described with reference to FIGS. 1 and 2.

Further examples of the pneumatically controlled valves are described in U.S. Pat. No. 8,932,544 issued on Jan. 13, 2015, and titled MICROREACTOR ARRAY, DEVICE COMPRISING AMICROREACTOR ARRAY, AND METHOD FORUSINGA MCROREACTOR ARRAY, the disclosure of which is hereby incorporated by reference in its entirety for all purposes and specifically for description of the pneumatically controlled valves.

FIG. 13 is a conceptual diagram 1300 illustrating per well pH measurement and associated pH control for a plurality of microplate wells. As part of a protocol, one or more process parameters including at least pH are measured. In one cycle, the process parameters of each well of at least a subset of the wells of a microplate can be measured (e.g., a subset being identified by the protocol). For example, as shown in diagram 1300, there are N wells in the microplate and in the cycle each of the N wells are measured.

At a start of the cycle, the optical sensor device 212 of the pH measurement system 116 is positioned by the positioning device 114 in alignment with a first well 1302 and used to measure the pH in the first well 1302 at operation 1304. Based on the measurement, a determination is made at operation 1306 as to whether to adjust the pH in the first well 1302. The determination can be made based on whether a deviation or difference is identified from a predetermined pH identified by the protocol to the measured pH. If the determination is made to adjust the pH in the first well 1302, then the pH is adjusted at operation 1308 using the pH adjustment system 118 to correct the deviation.

Once the pH has been measured in the first well 1302 at operation 1304, along with one or more other process parameters to be measured, if any, the optical sensor device 212 of the pH measurement system 116 can be positioned by the positioning device 114 in alignment with a second well 1310 and used to measure the pH in the second well 1310 at operation 1312. The second well 1310 may be a next well that immediately follows the first well 1302 in a predefined measurement pattern. In some examples, operation 1312 occurs after at least operation 1306 has been partially performed with respect to the first well 1302. Based on the measurement, a determination is made at operation 1314 as to whether to adjust the pH in the second well 1310. Similar to operation 1306 with respect to the first well 1302, the determination made at operation 1314 can be based on whether a deviation or difference is identified from a predetermined pH identified by the protocol to the measured pH. If the determination is made to adjust the pH in the second well 410, then the pH is adjusted at operation 1316 using the pH adjustment system 118 to correct the deviation.

Such operations of measuring the pH, determining whether the pH is to be adjusted, and adjusting if so determined are repeated on a well-by-well basis following the predefined measurement pattern for each of the wells until the last well (e.g., the N^{th} well 1318) is reached. For example, the optical sensor device 212 of the pH measurement system 116 is positioned by the positioning device 114 alignment with the N^{th} well 1318 and used to measure the pH in the N^{th} well 1318 at operation 1320. In some examples, operation 1320 occurs after at least the determination of whether the pH is to be adjusted has been partially performed with respect to an immediately preceding well. Based on the measurement, a determination is made at operation 1322 as to whether to adjust the pH in the N^{th} well 1318. Similar to operation 1306 with respect to the first well 1302 and operation 1314 with respect to the second well 1310, the determination made at operation 1322 can be based on whether a deviation or difference is identified from a predetermined pH identified from the protocol to the measured pH. If the determination is made to adjust the pH in the N^{th} well 1318, then the pH is adjusted at operation 1324 using the pH adjustment system 118 to correct the deviation. The cycle then ends. In some examples, the cycle can be iteratively repeated every set interval of time.

The various examples and teachings described above are provided by way of illustration only and should not be construed to limit the scope of the present disclosure. Those skilled in the art will readily recognize various modifications and changes that may be made without following the examples and applications illustrated and described herein, and without departing from the true spirit and scope of the present disclosure.

What is claimed is:

1. A system for controlling pH in parallel culture wells, the system comprising:
a pH measurement system that, during a cycle, successively measures pH for each of a plurality of culture wells contained in a microplate;
a pH adjustment system that, during the cycle, adjusts the pH in one or more of the plurality of culture wells; and
a controller communicatively coupled to the pH measurement system and the pH adjustment system, the controller comprising a processing device and a memory coupled to the processing device and storing instructions that, when executed by the processing device, causes the controller to:
receive, from the pH measurement system, a measured pH value for a first culture well of the plurality of culture wells; and
initiate pH control for the first culture well before receiving, from the pH measurement system, one or more additional measured pH values for one or more other culture wells of the plurality of culture wells, wherein the pH control comprises:
comparing the measured pH value in the first culture well to a predetermined pH value for the first culture well:
based on the comparison, determining whether the measured pH value deviates from the predetermined pH value; and
in response to a determination that the measured pH value deviates from the predetermined pH value, generating and providing a signal to the pH adjustment system to cause the pH adjustment system to adjust the pH in the first culture well to correct the deviation.

2. The system of claim 1, wherein the microplate is insertable into one of a bioreactor or a microbioreactor.

3. The system of claim 1, wherein the microplate includes 32 to 48 culture wells.

4. The system of claim 1, wherein the pH measurement system comprises:
an optical sensor device comprising an optical source and a sensor; and
a pH optode immobilized in one or more culture wells of the plurality of culture wells.

5. The system of claim 1, wherein a range of pH measurable by the pH measurement system is from 3.6 to 8.

6. The system of claim 1, wherein the pH adjustment system comprises:
a fluid source containing a pH adjusting fluid; and
a fluidics device for conveying the pH adjusting fluid from the fluid source to each of the plurality of culture wells.

7. The system of claim 6, wherein the fluidics device is integrated with the microplate and includes:
a plurality of channels connecting the fluid source to each of the plurality of culture wells to convey the pH adjusting fluid from the fluid source to each of the plurality of culture wells; and
a plurality of valves controlling a flow of the pH adjusting fluid between the fluid source and the plurality of culture wells via the plurality of channels.

8. The system of claim 7, wherein the plurality of channels are disposed planarly underneath a bottom surface of the microplate to allow the pH adjusting fluid to be conveyed from the fluid source to each of the plurality of culture wells via an opening in a bottom surface of each of the plurality of culture wells.

9. The system of claim 7, wherein the microplate includes a cover and the plurality of channels are included in the cover to allow the pH adjusting fluid to be conveyed from the fluid source to each of the plurality of culture wells via an opening in a top surface of each of the plurality of culture wells.

10. The system of claim 7, wherein the fluidics device is a microfluidic chip comprising the plurality of channels and the plurality of valves, and wherein the microplate further includes a plurality of reservoir wells as the fluid source containing the pH adjusting fluid, the plurality of reservoir wells positioned above the plurality of valves.

11. The system of claim 6, wherein a top surface of each of the plurality of culture wells include one or more apertures and the fluidics device is an automated pipetting system to convey the pH adjusting fluid from the fluid source to each of the plurality of culture wells via the one or more apertures.

12. The system of claim 6, wherein to adjust the pH in the first culture well to correct the deviation, the pH adjustment system is caused to dose the first culture well with a particular volume of the pH adjusting fluid from the fluid source via the fluidics device, the particular volume based on a correcting variable computed by the controller.

13. The system of claim 6, wherein the pH adjusting fluid is one of a liquid actuator or carbon dioxide gas.

14. The system of claim 1, wherein the controller is further caused to:
receive, from an application associated with the system that is executing on a computing device, a protocol generated based on user input; and
store the protocol in the memory.

15. The system of claim 14, wherein the predetermined pH value for the first culture well is obtained from the protocol, the protocol including pH profiles corresponding to the plurality of culture wells, wherein the pH profiles include expected pH values for the plurality of culture wells.

16. The system of claim 15, wherein at least two of the pH profiles corresponding the plurality of culture wells are different from one another.

17. The system of claim 15, wherein a pH profile for at least one of the plurality of culture wells includes a plurality of expected pH values, each of the plurality of expected pH values corresponding to a time period.

18. The system of claim 15, wherein the controller is further caused to:
receive an instruction corresponding to a user input for modifying a portion of the protocol as an experiment is being run in accordance with the protocol; and
cause, in response to the received instruction, the portion of the protocol to be modified based on the received instruction as the experiment is being run, the portion of the protocol modified including at least one pH profile of one of the plurality of culture wells.

19. The system of claim 14, wherein the pH of the plurality of culture wells is one process parameter defined by the protocol for measurement, and the protocol further defines one or more additional process parameters for measurement, the one or more additional process parameters including at least one of dissolved oxygen, biomass, and fluorescence intensity.

20. The system of claim 14, wherein the protocol defines a time interval between successive measurements of pH, by the pH measurement system, for the plurality of culture wells.

21. The system of claim 1, further comprising:
a positioning device communicatively coupled to the controller that positions the pH measurement system from culture well to culture well during the cycle to enable the pH measurement system to successively measure pH for each of the plurality of culture wells.

22. The system of claim 21, wherein the controller is further caused to:
after receiving, from the pH measurement system, the measured pH value for the first culture well, generate and provide a signal to the positioning device to cause the positioning device to reposition the pH measurement system to a next culture well of the plurality of culture wells for which pH is to be measured.

23. The system of claim 1, wherein the pH measurement system successively measures pH for each of the plurality of culture wells in a predefined pattern.

24. The system of claim 23, wherein the predefined pattern is a serpentine pattern.

25. The system of claim 23, wherein the predefined pattern is a raster pattern.

26. A method for controlling pH in parallel culture wells, the method comprising:
measuring pH in a culture well of a plurality of culture wells contained in a microplate; and
initiating pH control for the culture well before measuring pH in at least one or more other culture wells of the plurality of culture wells, wherein the pH control comprises:
comparing the measured pH in the culture well to a predetermined pH for the culture well;
based on the comparison, determining whether the measured pH deviates from the predetermined pH; and
in response to a determination that the measured pH deviates from the predetermined pH, adjusting the pH in the culture well to correct the deviation.

27. The method of claim 26, wherein adjusting the pH in the culture well to correct the deviation comprises adjusting the pH in the culture well within about 10 seconds of measuring the pH in the culture well.

28. The method of claim 26, wherein adjusting the pH in the culture well to correct the deviation comprises dosing the culture well with pH adjusting fluid.

29. The method of claim 28, further comprising:
determining a volume of the pH adjusting fluid to dose the culture well with based on a computed correcting variable.

30. The method of claim 29, further comprising:
computing the correcting variable based on a difference between the predetermined pH and the measured pH, a proportional factor of a proportional component, and a proportional factor of an integral component.

31. The method of claim 26, wherein the pH is one process parameter measured in the culture well, and the method further comprises:
measuring one or more additional process parameters in the culture well before measuring the pH in the at least one or more other culture wells of the plurality of culture wells, the one or more additional process parameters including at least one of dissolved oxygen, biomass, and fluorescence intensity.

32. The method of claim 26, further comprising:
determining whether the deviation of the measured pH from the predetermined pH meets a threshold value, wherein the pH in the culture well is only adjusted if the threshold value is met.

33. One or more computer readable non-transitory storage media storing instructions that, when executed by at least one processing device, cause the at least one processing device to:
receive, from a pH measurement system, a measured pH value for a culture well of a plurality of culture wells contained in a microplate; and
initiate pH control for the culture well before receiving, from the pH measurement system, one or more additional measured pH values for one or more other culture wells of the plurality of culture wells, wherein the pH control further causes the at least one processing device to:
compare the measured pH value in the culture well to a predetermined pH value for the culture well;
based on the comparison, determine whether the measured pH value deviates from the predetermined pH value; and
in response to a determination that the measured pH value deviates from the predetermined pH value, generate and provide a signal to a pH adjustment system to cause the pH adjustment system to adjust the pH in the culture well to correct the deviation.
